(19) United States
(12) Patent Application Publication
(10) Pub. No.: US 2023/0165258 A1
(43) Pub. Date: Jun. 1, 2023

(54) BIOCIDAL POLYURETHANE SYSTEMS, METHODS FOR THEIR PREPARATION AND USES THEREOF

(71) Applicant: X-Infex B.V., Bussum (NL)

(72) Inventors: Herbert Wilhelm ULMER, Bussum (NL); Nico Hendrik KLAY, Bussum (NL)

(21) Appl. No.: 17/998,053

(22) PCT Filed: May 7, 2021
(86) PCT No.: PCT/NL2021/050298

§ 371 Date: Nov. 7, 2022

(30) Foreign Application Priority Data
May 8, 2020 (EP) 20173685.7

(51) Int. Cl. A01N 59/12; C08L 75/08; C08G 18/73; C08G 18/76; C08G 18/48; C08G 18/32; C08G 18/66; C08G 18/20; C08K 3/02; C09D 175/08; C09D 5/14; C09D 7/65; C09D 7/61; C09D 5/16; A01N 25/10; A01P 1/00

(52) U.S. Cl. CPC A01N 59/12; C08L 75/08; C08G 18/73; C08G 18/7614; C08G 18/4833; C08G 18/3206; C08G 18/6677; C08G 18/2081; C08K 3/02; C09D 175/08; C09D 5/14; C09D 7/65; C09D 7/61; C09D 5/1637; C09D 5/1662; A01N 25/10; A01P 1/00; C08L 2205/025; C08L 2203/30

(57) ABSTRACT

The invention relates to the field of polymers, in particular to polymer systems based on polyurethane (PU) having abroad spectrum biocidal activity and the use thereof in the manufacture of biocidal products. Provided is a process to provide a biocidal polyurethane-iodin e (PU-I) complex, comprising (i) dissolving at least one iodine source into one or more raw materials used for preparing the desired polyurethane (PU) to obtain a single phase iodine system, followed by (ii) conducting a PU polymerization reaction in the presence of the single phase iodine system to generate a biocidal PU-I complex in situ.

The invention relates to the field of polymers, in particular to polymer systems based on polyurethane (PU) having a broad spectrum biocidal activity and the use thereof in the manufacture of biocidal products. More in particular, it relates to polyurethane-iodine (PU-I) complexes including their manufacturing process, their mechanical and biocidal properties and various applications of said PU-I materials.

Polyurethanes (PUs) are used across a broad array of markets and applications. The materials can be thermosetting or thermoplastic, rigid and hard or flexible and soft. The materials can be easily extruded and molded into almost an unlimited number of shapes and forms, including coatings, filaments, sheets, molded parts, fibers and foams. The materials have good hardness, tensile strength, compression strength, impact resistance, abrasion resistance and tear strength. PUs are used in a broad range of applications including: filters, foams, insulation, wound coverings, catheters, foam seating, mattresses, rigid foam insulation panels, seals and gaskets, durable elastomeric wheels, bushings, electrical coverings, panels, adhesives, surface coatings and sealants, synthetic fibers, carpet underlay, hard plastic parts, condoms, hoses, air and liquid filters, flexible tubing, medical devices and food packaging to name a few. The PU materials can be further blended with a host of other natural and synthetic rubbers and polymers to produce blends and interpenetrating polymer networks for use in related and additional applications.

The urethane group is traditionally formed by reacting an alcohol with an isocyanate. PUs are typically made by the reaction of a polyol with an isocyanate. The reaction is schematically indicated below.

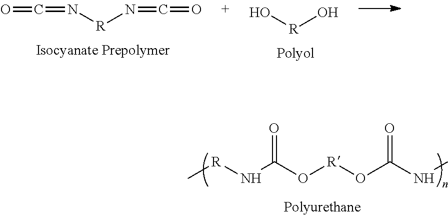

Thus, the urethane group is traditionally formed by reacting an alcohol with an isocyanate. Polyurethanes are formed when alcohols having functionality ≥2 are reacted with isocyanates having functionality ≥2 to generate an alternating copolymer. In order to better define and differentiate the types of alcohol used in polyurethane reactions, the following terms are often used: polyol, chain extender and crosslinker.
A polyol generally refers to a larger molecular weight, polymeric end-capped hydroxyl compound having functionality ≥2, while chain extenders are low molecular weight hydroxyl and amine terminated compounds having functionality=2 and crosslinkers being low molecular weight hydroxyl and amine terminated compounds having functionality ≥3. The following table gives some examples of types of “alcohols” used in polyurethane reactions.

| | | Functionality |
|---|---|---|
| | | (hydroxyl |
| Designation | Structure | groups) |
| Polyether polyol | 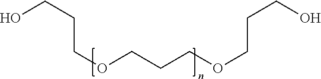 | 2 |
| Polyester polyol | 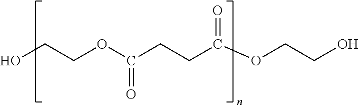 | 2 |
| Ethylene glycol chain extender | 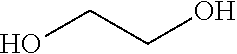 | 2 |
| 1,4 Butanediol chain extender | 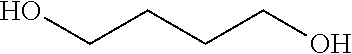 | 2 |
| Glycerol | 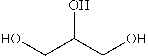 | 3 |
| Pentaerythritol cross linker | 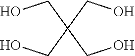 | 4 |

Whereas the actual combination of hydroxyl-containing compounds that can be mixed and reacted to give a polyurethane are almost endless, the formation of the urethane linkage is the same for all compounds: the reaction of the alcohol hydroxyl group with an isocyanate.
Acceptable isocyanates are also available in a range of functionalities, including diisocyanates (functionality=2), triisocyanates (functionality=3) and polyisocyanates (functionality >3). They are available as either aliphatic or aromatic isocyanates.
A distinction can be made between thermoplastic polyurethanes (TPU) and thermoset polyurethanes. Thermoplastic polyurethanes are materials that form physical crosslinks between phases in the polyurethane that can be “melted” upon applying heat or solvent. These polymers can be molded via injection molding and extruded via hot melt extrusion. The materials can also be dissolved in solvents. The reason that the materials can be “melted” is that they are not chemically crosslinked because the polymerization reaction is generally between diols and diisocyanates that both have a functionality of two. This way, a linear polyurethane is formed. Thermoplastic urethane is a polymer that can be melted and reformed, and is elastic and highly flexible, making it a versatile material suitable for use in a wide range of industries.
Thermoset polyurethanes are chemically cross linked, and the resultant materials cannot be “melted” and reformed. Thermoset PU is generally more durable than thermoplastic urethane. The reason for this is that the functionality of the alcohol and/or isocyanate systems used possess a functionality >2. The use of higher functionality raw materials (functionality >2) increases the likelihood for chemically linking polymer chains until an infinite crosslinked structure is obtained that cannot be degraded by heat or dissolution.
Often, polyurethane foams are desired which requires the addition of a blowing agent and surfactant. Blowing agents can be volatile liquids such as low boiling hydrocarbons or hydrofluorocarbon or more preferably an inert gas. Carbon dioxide is especially preferred as a blowing agent, which can be added directly as a gas or generated by the addition of water to the polyurethane reaction. Water quickly reacts with the isocyanate to generate an amine and carbon dioxide. The resultant amine further reacts with more isocyanate to give an urea linkage in the polymer with the resultant carbon dioxide acting as a blowing agent to produce the polyurethane foam. The schematic for this process is indicated below.

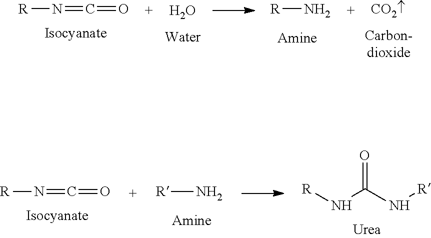

Thus, water addition to the PU reaction results in the generation of both urethane and urea linkages in the final foam matrix.
Whereas PUs have excellent mechanical properties, the materials are not biocidal and micro-organisms readily populate PU surfaces. PUs are susceptible to microbial attack and this is considered one of the major drawbacks for this large class of polymers.

Bacterial growth on industrial our household PUs is annoying and undesirable, and bacterial growth on PUs for healthcare applications can be deadly. The article, Antimicrobial strategies to reduce polymer biomaterial infections and their economic implications and considerations, International Biodeterioration & Biodegradation, 136 (2019) 1-14, describes the problem of Hospital Acquired Infections (HAI), how medical devices contribute to HAI's and the various strategies used to make polymers used in medical devices antimicrobial. Catheters are the most common implant worldwide, with 5 million central venous catheters (CVC) and 30 million urinary catheters being implanted per year, in the USA alone. With both catheter types identified as two of the leading origins of HAI's. The Centers for Disease Control (CDC) reported that approximately 250,000 CVC related bloodstream infections (BSI) occur each year in the USA alone at an additional hospitalization cost of ca. $34,500-$56,000 per each infection. Not only is there a huge economic cost, the CDC estimates that 4% of patients in the USA will contract a HAI's during their hospital stay, which will result in 1.7 million infections and 99,000 associated deaths.

This same article indicates that a relatively small number of microorganisms is responsible for the majority of the HAI's, mainly Staphylococcus aureus, Pseudomonas aeruginosa, Escherichia coli, coagulase-negative staphylococci (CoNS) (predominately S. epidermis) and Enterococcus ssp. (predominately E. faecalis and E. faecium). With vascular implants more likely to be colonized by S. aureus and CoNA, while E. coli and Enterococcus sp. are more likely to colonize urinary devices.

PUs are commonly used in a number of medical applications including both vascular and urinary catheters, wound dressings, tubing, medical device packaging, filters, hospital bedding, surgical drapes, feeding tubes, surgical drains, intra-aortic balloon pumps, dialysis devices, non-allergic gloves, medical garments, as well as a variety of injection molded devices. PUs most common use is in short-term implants, where the potential for microbial contamination is high. It would be expected that it would be a great benefit if these medical devices and health related products could be made biocidal to reduce the spread of undesirable pathogenic microorganisms, resulting in HAI's. HAI's are caused by the spread of viral, bacterial and fungal pathogens due to a healthcare visit or care (e.g. hospital, doctors office, nursing home, etc.). The most common ways of acquiring HAI's are via bloodstream infection, pneumonia, urinary tract infection and surgical site infection. PUs are responsible for the fabrication of a multitude of products that are in direct contact with: wounds, air passageway, blood and urinary tract and thus become a potential source for pathogenic contamination risk resulting in HAI's. If not a source, PUs can become a sight for the promotion of unwanted microorganism accumulation, which can lead to unwanted infections.

In view of the above, PU or PU-based materials having biocidal activity would be highly desirable for specific applications, especially applications that could improve consumer and patient health and reduce the risk of HAI's.

There are multiple patents and literature references that describe making PU materials biocidal by including antibacterial agents into the PU polymerization. These agents can be both reactive agents that are fixed to the PU structure and/or additives that are included into the PU matrix. Such bacterial agents include: quaternary amino compounds (e.g. benzalkonium chloride and cetylpyridinium chloride), phenols and cresols, halophenols (e.g. p-chloro-m-xylenol), biguanides (e.g. chlorhexidine), anilides (e.g triclocarbon), and triclosan

One class of antibacterial agents that has garnered significant attention and is responsible for a number of commercial systems in the use of metals such as: copper, zinc, silver and their corresponding derivatives and/or salts. These metal systems are either coated onto or blended into the polymer substrate. Nano-silver has been especially singled out as a desirable antibacterial metal that can render coated or extruded systems biocidal. While these systems are promising and have had some commercial success, there are also concerns with the use of metal and nano-metal systems. First, the metals have a potential for leaching and/or removal from the PU system. A study on washing nano-silver infused fabrics showed that the level of nano-silver removed from the clothing after just one wash was 20-35%. Second, the toxicity of metal and especially nano-metal systems is not completely understood. Nano-metal systems have the ability to cross the skin/cell barrier and thus there is concern on patient safety. The nano-metal systems are also easily leached or removed from treated systems and enter the ecosystem, where the effect of these metal nano-particles on the environment is unknown.

Related to antibacterial agents, there are also many references of making PU's antibacterial by adding known antibiotic agents to the PU system. Unfortunately, the wide use of antibiotics has led to the generation of a number of antibiotic resistant bacterial or “superbugs” as they are referred to. This development is especially troublesome since the potential risk of acquiring HAI's that cannot be treated has drastically increased, which has direct consequences to patients' health.

Based on the above, it is evident that the discovery of new biocidal PU materials and the products that use said biocidal materials that can combat existing and future antibacterial superbugs and viruses would be highly desirable.
A unique attribute of PUs is that the urethane linkage forms strong complexes with iodine. Iodine is a unique material in that it is a natural occurring and environmentally benign material that is readily available, inexpensive and shows strong antiseptic activity against bacteria, viruses and fungi, but is also a needed essential mineral nutrient for normal healthy body function. The actual complex mechanism is thought to be similar to the water-soluble polyvinylpyrrolidone-iodine (PVP-I) iodophor complex. PVP-I (betadine) is commercially available and is widely used as an antiseptic for skin disinfection before and after surgery and of minor wounds. The actual PVP-I complexation is depicted in the following scheme.

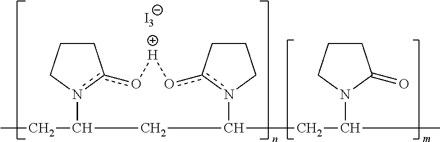

The general consensus is that the complex takes place via hydrogen bonding/charge complex between the amide functionality on the PVP and the iodine to form a donor-acceptor complex in which the iodine is the acceptor. A similar donor-acceptor complex is envisioned to take place with the urethane and urea groups in polyurethanes.

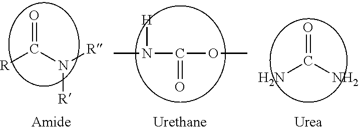

PVP-I is a water-soluble iodophor used extensively in the healthcare and veterinarian sector as an antiseptic. PVP-I was introduced in the 1950's and is made by complexing polyvinylpyrrolidone (PVP) with iodine (I). The PVP-I material is a stable complex that has similar broad-spectrum activity against microorganisms as iodine, but is less irritating and toxic. The poison label required for iodine products is not necessary for preparations containing PVP—I and commercial solution and salve-based formulations are readily available over the counter (OTC product). Solutions of PVP-I are non-irritating and non-sensitizing and cause no pain when applied to wounds or mucous membranes. PVP-I solutions can be used as a mouth rinse resulting in no staining or negative side effects. PVP-I solutions are active against: bacteria, bacterial spores, yeast, mold, fungi, viruses and bacteriophages. The broad-spectrum biocidal activity is especially effective at treating mixed infections. An overview of PVP-I can be found in the PVP-Iodine brochure issued by International Specialty Products, brochure Phar0019 3/2004. Conceivably, a polyurethane-iodine complex production process that is flexible, simple and cost effective to produce polyurethanes that have similar biocidal properties as the water soluble PVP-I and are safe, non-irritating, biocidal and antiviral would be of high interest.

U.S. Pat. No. 3,235,446 discloses how to prepare iodinated polyurethane foams and films. The initial PU foams are soaked in water-alcohol iodine solutions to slowly allow iodine to diffuse into the foam. A number of disadvantages prevents this process from being commercially viable: (1) long process times for the foam to adsorb the iodine solution, (2) need to dry the foam afterwards, (3) inhomogeneous uptake of the iodine throughout the foam, (4) the PU chemical structure of the foam has a significant effect on the ability to “absorb” the iodine solution.

U.S. Pat. No. 4,381,380 discloses how to prepare thermoplastic polyurethanes articles treated with iodine. Here the inventor takes prepared PU thermoplastic articles and treats them with water-alcohol iodine solutions.

U.S. Pat. No. 4,769,013 discloses biocidal coatings for PU's that are produced by initially treating the PU with an organic solvent solution containing PVP to attach the PVP polymer to the PU article and subsequently treating with an iodine solution to form an attached PVP-I biocidal coating.

U.S. Pat. No. 5,302,392 discloses a PU foam with rapid iodine release. The foams are produced by including PVP-I as solid powder in the PU polymerization process to form an interpenetrating network (IPN) of solid PVP-I particles dispersed in (i.e. not complexed to) the PU foam matrix. The patent discloses that the resultant foam contains solid particles of PVP-I that are substantially uncomplexed with the PU matrix. Adding an aqueous solution to the resultant PU—PVP-I IPN results in instantaneous release of the PVP-I complex into said liquid. U.S. Pat. No. 5,302,392 is not focused on making the PU foam material biocidal, but using the PU foam as a delivery device to deliver the water-soluble PVP-I complex when wetted. There is no reaction to make a PU-I complex and the water-soluble PVP-I quickly leaches from the PU matrix for immediate activity rather than providing a PU material having a long lasting and controlled biocidal activity.

Although all these prior art methods result in the production of a biocidal PU product, there are multiple issues and limitations which have thus far hampered their wide scale application.

First, all known manufacturing methods require the use of solvents to form the iodine complex. The PUs need to be placed in contact with organic solvent systems, soaked and then subsequently dried. These processes are time consuming, costly, energy intensive and not environmentally friendly. Large scale implementation of these technologies is therefore not economical or practical. Second, the process generally only works with hydrophilic PUs, where the PU will interact and swell in the alcohol-water iodine solutions to generate the complex. Hydrophobic PUs that have little or no interaction with polar solvent systems would not be expected to work. Third, the processes are very difficult to standardize to develop a commercial process in which the same material with the same iodine complexation is generated on a continuous basis. Even very small variations in the chemical and physical structure of PU would have a potentially large effect on the iodine uptake and complexation process. Finally, since all prior art methods rely on absorbing iodine from a solution onto the PU substrate—a surface application, it is very difficult to standardize the level, complexing efficiency and complex depth of the PU-I. It is almost guaranteed that significant biocidal variations and activity would be observed for the resultant PU-I systems, something which is not acceptable for regulated (medical care) products.

In view of the above, the inventors set out to provide a novel approach for the manufacturing of PU-I systems which overcomes at least some, preferably all of the prior art shortcomings mentioned above. In particular, they aimed at providing PU-I complexes that are simple to manufacture, stable, homogeneous and inherently biocidal, while avoiding the need of undesirable solvents and/or complicated manufacturing processes.
Quite unexpectedly, the present inventors have discovered that PU's can be complexed with a suitable iodine source in a process wherein an iodine source is dissolved directly in one or more raw materials used to manufacture the desired PU, such that the PU-I complex is formed “in-situ” during the PU polymerization reaction, or wherein an iodine source is added in a “melt” of a preformed PU wherein the PU is soluble and accessible for complexation of an iodine source with the urethane and urea groups in the PU matrix. The preformed PU can be a thermoplastic PU (TPU) or PU in an aqueous dispersion (PUD). For example, an iodine source is added via hot melt extrusion with TPU, or by allowing iodine migration to the dispersed PU phase in an aqueous continuous phase of a polyurethane dispersion (PUD). The resultant biocidal properties of the PU-I material can be easily tuned depending on the amount and/or source of iodine used in the processing.

### PU-I Complex Formation In Situ

One aspect of the invention relates to a process for providing a biocidal polyurethane-iodine (PU-I) complex, comprising (i) dissolving at least one iodine source into one or more raw materials used for preparing the desired polyurethane (PU) to obtain a single phase iodine system, followed by (ii) conducting a PU polymerization reaction in the presence of the single phase iodine system to generate a biocidal PU-I complex.
Without wishing to be bound by theory, in this process the PU-I complex is formed “in-situ” at about the same time as the urethane group to form a stable PU-I complex.
Such process is not disclosed or suggested in the art.
As mentioned herein above, U.S. Pat. No. 5,302,392 discloses the preparation of a PVP-I/PU foam wherein a slurry of dry powder PVP-I in polyethylene glycol is added to isocyanate reactant to start the PU foaming process. Unlike the present invention, the PVP-I is added to the PU reaction mixture as a binary iodine system. As a result, the polyurethane composition of U.S. Pat. No. 5,302,392 has particles of solid PVP-I complex evenly distributed throughout the polyurethane matrix so that the complex does not associate with the polyurethane to any appreciable extent. The composition is particularly useful as a sponge for scrubbing which provides almost instantaneous release of the complex.
As used herein, the term “raw material” refers to any precursor, reactant, or starting material that is conventionally used in the manufacture of a polyurethane.

The “single phase iodine system” or “one-phase iodine system” is meant to refer to a homogeneous solution of the at least one iodine source in any type or number of raw materials for the PU reaction. It is a homogeneous one-phase liquid mixture consisting of two or more components, at least one of which is the iodine source (solute) and at least one is a component (solvent) used in the subsequent PU reaction wherein the iodine source is soluble. It does not encompass a mixture of a solid or particulate fraction suspended in a liquid, such as a slurry or a suspension. For example, excluded are slurries of dry powder PVP-I in a polyol.

It is important to understand that the actual polyurethane reaction and formation of the urethane groups that forms the complex with iodine are the same for both polyurethane thermoplastics and thermosets. The driver for obtaining a thermoplastic or thermoset is based on the functionality of the raw materials used in the polyurethane reaction rather than a chemical difference (i.e. the same urethane reaction of alcohol with isocyanate takes places in both systems). Thus, the ability to form the PU-I complex in-situ during the urethane reaction is applicable to both thermoplastic and thermoset polyurethanes.

According to the present invention, the at least one iodine source may be selected from the group consisting of elemental iodine, polyvinylpyrrolidone-iodine (PVP-I), iodide salts, and any combination thereof. The PVP-I may contain 1-25% available iodine and 2-35% total iodine. Preferably, the at least one iodine source is elemental iodine, optionally combined with PVP-I. In a specific embodiment, elemental iodine is the sole iodine source.
As outlined herein above, the in-situ process of the invention is among others characterized in that the at least one iodine source is included in the PU reaction mixture in a dissolved state, more in particular wherein it is dissolved in one or more raw material(s), e.g. precursor, reactant, solvent, that is typically used in the manufacture of a PU, be it a thermoset or thermoplastic PU. Suitable raw materials for dissolving the iodine source include (i) polyols; (ii) isocyanates; and (iii) a chain extender, crosslinker, catalyst, surfactant, solvent and/or additive used in the synthesis of polyurethane.
The invention thus relates to a process to provide a biocidal polyurethane-iodine (PU-I) complex, comprising dissolving at least one iodine source into polymerization mixture comprising raw material(s) for preparing a polyurethane thermoset or polyurethane thermoplastic; and allowing for formation of a biocidal PU-I complex. For example, this process suitably comprises dissolving at least one iodine source in a polymerization mixture comprising (i) a polyol and (ii)isocyanate and/or small molecule chain extender commonly used in the fabrication of polyurethane to provide a thermoset or thermoplastic polyurethane-iodine complex. It is preferred that either synthetic or bio-based starting materials are used to produce a polyurethane.

In one embodiment, at least one iodine source is first dissolved in an “incomplete” reaction mixture after which the reaction mixture is completed to initiate the PU reaction. For example, provided is a process wherein at least one iodine source, e.g. PVP—I and/or I₂, is dissolved in a polyol, a polyol blend, a low molecular weight alcohol with functionality ≥2, a low molecular weight amine with functionality ≥2 and/or solvent, followed by the addition of the desired isocyanate(s) to initiate the polyurethane reaction.

In another embodiment, the at least one iodine source is dissolved in the “complete” PU reaction mixture. For example, the iodine source(s) is/are dissolved in a polymerization mixture comprising (i) polyol; (ii) isocyanate; and (iii) a chain extender, crosslinker, catalyst, surfactant, solvent and/or additive used in the synthesis of the polyurethane to provide a thermoplastic or thermoset polyurethane-iodine complex.

Suitable isocyanates for use in the present invention are known in the art. The isocyanate may comprises an aliphatic di-, tri- or polyisocyanate, an aromatic di-, tri- or polyisocyanate, or any combination thereof. In a specific aspect, the isocyanate comprises an aliphatic or an aromatic di- or multifunctional isocyanate.

Suitable polyols for use in the present invention are also known in the art. For example, the polyol is selected from the group consisting of polyether polyol, polyester polyol, polycarbonate polyol, polycaprolactone polyol, polyacrylate polyol and any combination thereof.

The chain extender can be a low molecular weight diol or diamine, or any combination thereof.

The crosslinker can be a low molecular weight alcohol or amine with a functionality of more than 2.

The polyurethane catalyst can be any conventional or yet to be discovered PU catalyst, for example a tertiary amine, metallic compound, or any combination thereof. It was observed that the PU polymerization reaction in the presence of the single phase iodine system can be inhibited by the iodine species. Therefore, in a preferred embodiment increased amounts of catalyst is added in order to proceed with the polyurethane reaction and PU-I complex formation.

According to the invention, in-situ PU-I complex formation may involve conducting the polyurethane polymerization reaction via multi-step, one-step bulk or solvent polymerization to form the final PU-I complex in pre-polymer formation stages or one process step.

PU-I Complex Formation with Preformed PU

The invention also relates to a process wherein an iodine source is complexed to a preformed PU by using specific conditions to ensure that the PU is accessible to complexation with an iodine source. Provided is a process to provide a biocidal polyurethane-iodine (PU-I) complex, comprising preparing a homogeneous mixture of (i) at least one iodine source and (ii) a thermoplastic polyurethane (TPU) or a PU dispersion (PUD) to form a single-phase system that allows for formation of a biocidal PU-I complex.

So, similar to what is described herein above for the in-situ process, the single phase system ensures that the existing PU structure is in its “melt” physical state that allows for an interaction (complexation) with the iodine source(s).

TPU in a melt physical state can be achieved in various manners. For example, TPU can be heated above its glass and/or crystallization temperature, or it is dissolved in a suitable solvent. Accordingly, the process may comprise preparing a homogeneous single-phase system by blending at least one iodine source with TPU in a heated, molten or dissolved state. This process may comprise dissolving at least one iodine source in a melt or solution of a thermoplastic polyurethane to provide a thermoplastic polyurethane-iodine (TPU-I) complex. Also encompassed is a process to use TPU-I complex as a master batch that is further processed into a given product.

In one embodiment, it comprises mixing an iodine source and TPU as dry powders, and feeding the mixture into an extruder. In a specific aspect, elemental iodine and/or PVP—I is/are dry mixed with TPU, followed by extrusion.

The process can be put into practice with any type of TPU. For example, the thermoplastic polyurethane comprises one or more of polyester-based, polyether-based, polycaprolactone-based, polyacrylate-based, aromatic and/or aliphatic thermoplastic polyurethanes. Preferably, a polyurethane that is produced from either synthetic or bio-based starting materials.

It may be advantageous that TPU is the major or dominant polymer in the homogeneous mixture of (i) at least one iodine source and (ii) a thermoplastic polyurethane (TPU). In one aspect, TPU makes up at least 85 w %, preferably at least 90 w %, more preferably at least 95 w % or even at least 98 w % of the total polymer content of the homogeneous mixture. It may be preferred that the homogeneous mixture does not contain any hydrophilic polymer(s), such as poly(N-vinyl lactam).
Similar to what is described herein above for the in-situ process, the at least one iodine source can be selected from the group consisting of elemental iodine, polyvinylpyrrolidone-iodine (PVP-I), iodide salts, and combinations thereof.
In one embodiment, at least PVP—I is used as iodine source. In a preferred embodiment, PVP—I contains 1-25% available iodine and 2-35% total iodine.
Preferably, use is made of a PVP-I that conforms to the USP or EP pharmacopeias for povidone-iodine. In another embodiment, a combination of PVP-I and elemental iodine is used, for example in a hot melt extrusion process for preparing the PU-I complex. In a preferred aspect, the at least one iodine source is or comprises elemental iodine.
As is exemplified herein below, the invention provides a process for the manufacture of a biocidal polyurethane-iodine (PU-I) complex, for instance to be used as biocidal coating, wherein the preparation of a homogeneous iodine/PU mixture comprises adding elemental iodine to an aqueous polyurethane dispersion (PUD) and allowing migration of the elemental iodine into the PU phase of the dispersion to obtain a homogeneous single-phase system wherein the PU-I complex is formed.
Aqueous polyurethane dispersions are known in the art, and readily available from commercial suppliers. For example, aliphatic polyether polyurethane dispersions can be obtained from Rudolf GmbH, Geretsried, Germany.

In one embodiment, elemental iodine is added to the PUD as a solution in a suitable (non-aqueous) solvent that dissolves the elemental iodine and is compatible with the PU phase, such as an alcohol, preferably isopropanol. See also Example 2 and FIG. 1A. In another embodiment, elemental iodine is added as solid material, followed by iodine sublimation. Iodine readily sublimes at room temperature, but this can be catalyzed by applying heat. See Example 2 and FIG. 1B. In yet another embodiment, elemental iodine is dissolved in an excess amount of alcoholic solvent and added to an aqueous PUD to form a single-phase hydroalcoholic solution that allows for formation of a biocidal PU-I complex. See Example 2 and FIG. 1C.

### Biocidal PU-I Complexes and Applications Thereof

The invention also relates to a biocidal polyurethane-iodine complex that is active against e.g. bacteria, viruses, yeasts, fungi, mold, spores and/or protozoa. In particular, it provides a PU-I complex obtainable by a process according to the invention. The PU-I complex is either prepared by an in-situ method according to the invention, or by iodine complexing to preformed TPU under the conditions described herein above. The PU-I complex is characterized among others by the absence, or virtually non-detectable release or leaching of iodine from the PU material. The PU-I complex may comprise thermoplastic PU (also referred herein as TPU-I) or thermoset PU or aqueous PUD.
In one aspect, the invention provides a polyurethane-iodine complex comprising 1-30 weight % PVP-I, like 2-15 w % PVP-I, 3-10 w % PVP-I or 3-8 w % PVP-I. I
In another aspect, the invention provides a polyurethane-iodine complex comprising 0.1-30 weight % elemental iodine (I2), like 0.1-20 w %, 1-15 w % or 3-8 w % elemental iodine. In a preferred embodiment, the PU-I complex comprises 0.1-10 weight % elemental iodine. For example, provided is a polyurethane-iodine complex that possesses a minimum amount of solubilized iodine of 0.1 weight percent in the finished polyurethane-iodine complex material.
As will be appreciated by a person skilled in the art, a PU-I complex provided herein can be prepared or formulated in any suitable form, composition or material. These include foams, dispersions, coatings, solid articles, and the like. Preferably, PU represents the major polymer. In one embodiment, TPU makes up at least 85 weight %, preferably at least 90 weight %, more preferably at least 95 weight % of the total polymer content of the PU-I containing material. Alternatively or additionally, the material does not contain hydrophilic polymer(s), preferably no poly(N-vinyl lactam).
The PU-I materials of the invention are functionally characterized by the combination of desired mechanical properties and broad spectrum biocidal and antiviral activity against: bacteria, mold, viruses and fungi. The materials are non-toxic, non-irritating and non-sensitizing and have broad use applications in health, consumer, food, veterinarian, aquatic and industrial applications.
The invention therefore also provides the use of a polyurethane-iodine complex according to the invention in a large variety of (biocidal) applications, including the area of industrial, construction, consumer, pharmaceutical, health, veterinarian and/or aquatic markets. Such use may involve a blend, composite and/or interpenetrating network of the polyurethane-iodine complex with other natural or synthetic polymers, natural or synthetic fibers, biocidal agents and/or fillers.
As is evident from the above, the invention also relates to a biocidal product (i.e. an end product or consumer product) comprising a polyurethane-iodine complex as provided herein. Also here, the polyurethane-iodine complex may be part of a blend, composite and/or interpenetrating network with one or more other natural or synthetic polymers, natural or synthetic fibers, biocidal agents and/or fillers. Exemplary biocidal products include air filters, water and solution filters, mouth caps, gloves, equipment or device housing, adhesives, garments, curtains, fibers, hard surface coatings, dentistry articles, building materials, construction materials, carpets, medical devices, wound dressing, tissue scaffolds, operating and endo-scopes, catheters, tubes, breathing tubes, endotracheal tubes, intravascular catheters, deep intravenous lines, footwear, sponges, cutting planks, masks, hoses, food and device packaging, countertops, flexible surface coatings, key boards, upholstery, floor coatings, flooring, condoms, elastics, cardiac valves, pacemakers, mats, sealants, breast implants, implants, foams and gaskets.
Also provided is an aqueous dispersion comprising a biocidal PU-I complex according to the invention, and the use thereof in a method for providing a surface or object with a biocidal coating. In a further embodiment, the invention provides a biocidal coating comprising or consisting of a PU-I complex according to the invention. Also encompassed are objects, such as face masks, that are at least partially provided e.g. by spray coating, with biocidal PU-I complex.

### DETAILED DESCRIPTION

Due to the fact that the complexing can be conducted on a broad range of PUs having unique and desired properties, the potential applications for said PU-I complex articles represent the same applications that PU's are presently used with the added benefit of being biocidal. In addition, due to the fact that the PU-I complexes are manufactured in the melt, the resultant materials are inherently biocidal throughout the entire polymer matrix. The PU-I systems show broad spectrum biocidal activity against bacteria, viruses, yeasts, fungi, mold and protozoa. The PU-I materials are non-selective in their germicidal activity and can be used where PU materials having bactericide, fungicide, viricide, sporicide, amebicide, insecticide or nematocide activity are desired. The resultant PU-I complex and blend products: have low animal and phytotoxicity, are non-irritating to skin and mucous membranes, are non-sensitizing, [symbol] do not delay healing or formation of granulation tissue and are non-stinging. The PU-I complexes from this invention show long term biocidal activity.

The PU-I complex materials disclosed in this invention are expected to have similar broad-spectrum biocidal activity to the water-soluble PVP-I complex. PVP-I solutions are active against: bacteria, bacterial spores, yeast, mold, fungi, viruses and bacteriophages. PVP-I materials show broader biocidal activity than other antibacterial agents such as benzalkonium chloride and chlorhexidine. Unlike many antibiotics, there has been no bacterial resistance observed for PVP-I systems after more than 60 years of clinical use. PVP-I solution is active against methicillin-resistant Staphylococcus aureus (MRSA), Klebsiella pneumoniae, Streptococcus pneumoniae, Hemophilus influenzae, Pseudomonas aeruginosa and Legionella pneumophila and readily inactivates the human immunodeficiency virus (HIV) and polio virus. PVP-I solutions demonstrate high virucidal efficacy against Avian influenza (Virology J., 2009: 6: 124), the Ebola virus and the vaccinia virus Ankara used as a reference virus to show viricidal activity against enveloped viruses (BMC Infect Dis., 2015; 15: 375). PVP—I has also been shown to be highly active against the SARS coronavirus, SARS-CoV (Dermatology, 2006; 212 (suppl 1): 119-123), a coronavirus that is of the same family as the virus responsible for the global pandemic outbreak of COVID-19 and following global economic crisis.

PVP-I is obtained as a reddish-brown free flowing powder. It is usually obtained having an available iodine content of about 9-12%. The material is included in the USP and EP pharmacopeias. U.S. Pat. No. 4,200,710 gives the details of how such PVP-I complexes can be prepared and there are a number of manufacturers of PVP-I around the world, including Ashland Inc., BASF and Boai NKY Pharmaceuticals Ltd. Suitable PU and/or PU raw materials for use in the present invention include those that are produced by a number of global manufacturing companies, including: BASF SE, Bayer, Covestro, Lubrizol, DSM, Dow, BorsodChem, Elastogran, Huntsman Polyurethanes, LyondellBasell, Repsol, Emery Oleochemicals and Shell Chemicals. The materials include PU thermoplastics and the raw materials used to manufacture PU thermosets, mainly polyols and/or isocyanates.

According to the invention, a PU-I complex can be made by dissolving the desired iodine source into the PU resultant structure without the need for additional solvents. This is done for example by (1) dissolving the iodine source into a melted phase PU, e.g. TPU; (2) dissolving the iodine source in one or more starting raw materials, e.g. the polyols and/or the isocyanates, used to produce a PU; or (3) adding an iodine source to an aqueous polyurethane dispersion (PUD).
Common commercial aromatic isocyanates include: toluenediisocyanate (TDI), diphenylmethane diisocyanate (MDI), naphthalene diisocyanate (NDI), triphenylmethane triisocyanate and polymeric forms of MDI and TDI. Common commercial aliphatic isocyanates include: hexamethylene diisocyanate (HDI), isophorone diisocyante (IPDI) and hydrogenated MDI (HMDI).

In addition to said commercial isocyanates, a host of specialty di- and multifunctional isocyanates can be employed in this invention. Modified multifunctional isocyanates which are products obtained by the partial chemical reaction of organic isocyanates and/or multi-functional isocyanates may be used. Examples include, without limitations, diisocyanates and/or multi-functional isocyanates containing ester groups, ether groups, urea groups, biuret groups, allophanate groups, carbodiimide groups, isocyanurate groups, and/or urethane groups.

The polyol component may be any conventional polyol used to form polyurethanes. Exemplary polyols include polyhydroxy-containing polyesters, polyoxyalkylene polyether polyols, polyhydroxy-terminated polyurethane polymers, polyhydroxy-containing phosphorus compounds, and alkylene oxide adducts of polyhydric polythioesters, polyacetals, aliphatic polyols and thiols, and mixtures thereof. Commercial polyether polyols include: hydroxyl-terminated polypropylene oxide (PPO), polypropylene glycols (PPG), hydroxyl-terminates polyethylene oxide, polyethylene glycols (PEG) and polyols of polytetramethylene oxide (PTMO) and polytetrahydrofurane (PTHF). Commercial polyester polyols are often made from adipic acid and ethylene glycols (polyethylene adipate), or from butanediols and adipic acid (polybutylene adipate). In addition, polyester polyols can be prepared from a mixture of glycols and adipic acid to control the mechanical properties of the resultant polyol. Another important polyol is polycaprolactone diol, because it is considered biodegradable. Polyols can also be made by copolymerizing caprolactone with other monomers. There has also been considerable efforts to develop bio-based polyols to improve sustainability.

The PU compositions may further include chain extenders. The chain extenders may be aromatic or aliphatic compounds capable of reacting with at least two isocyanate terminated polymer units to form a polymer chain. Exemplary chain extenders may be aromatic or aliphatic compounds which are terminated with more than one hydroxyl or amine groups. Additional PU components can include: catalysts, surfactants, stabilizers, dyes, thickeners, plasticizers, fillers and pigments. The PU family of polymers represents one of the most broad and diverse class of synthetic polymers. PU can also be readily blended or composites constructed with other polymers and substrates to further broaden the scope of obtainable properties and applications.

Very good results can be obtained when preparing a homogeneous mixture of (i) at least one iodine source and (ii) a thermoplastic polyurethane (TPU) or a polyurethane dispersion (PUD) to form a single-phase system that allows for formation of a biocidal PU-I complex. As already indicated, the PU-I biocidal materials of the invention can be prepared without using solvents. For thermoplastic polyurethane (TPU) systems, the TPU-I complex may be formed by blending the TPU polymer with a suitable iodine source in the molten state. The TPU-I complexes are easily and effectively prepared via hot melt extrusion with the finished product manufacture completed by extrusion, injection molding or 3-D printing. Hot melt extrusion can be responsible for melt mixing and preparation of the TPU-I complex and fabrication of finished products or can be used to prepare a master batch that is used in further downstream processing and product production. Preferred temperatures to run the complexation depends on the TPU base material and the iodine substrate, with a preferred minimum extrusion temperature above the melt temperature of iodine, 114° C., in order to quickly and efficiently mix the iodine within the TPU matrix. The actual TPU-I extruding conditions are very dependent on the TPU material being blended with the iodine and PVP-I materials. Acceptable TPU materials that can be blended in the hot melt with iodine and/or PVP—I are any TPU that can be melted and extruded and include, but not limited to: polyester-based TPUs, polyether-based TPUs, caprolactone-based TPUs, aromatic TPUs and aliphatic TPUs. The TPUs are block copolymers consisting of alternating soft and hard segments that can be controlled to form an almost endless number of TPUs. The TPU block copolymer consists of: more polar, hard segments formed by the reaction of diisocyanates with short-chained diols (chain extenders) and less polar, soft segments formed by the reaction of diisocyanates with long-chained diols. By adjusting the ratio of soft and hard segments, TPUs can be synthesized encompassing a huge range of polymer properties with varying melt characteristics and mechanical properties. The soft and hard segments segregate into unique phases that results in crystalline or pseudo-crystalline phases to physically cross-link the TPU when cool. Heating the TPUs causes these “pseudo-crosslinks” to disappear and the TPU melts. It is during this melting process that the iodine source can be effectively complexed to the TPU matrix. Upon cooling, the hard and soft segments begin to phase separate and the TPU-I complex undergoes physical crosslinking to “set” the biocidal TPU-I complex in the desired form and shape. Because the resultant TPU-I can be melted, the TPU-I materials can be easily processed via: extrusion, injection molding, blow molding, fabric coating, over molding, calendaring, compression molding, vacuum-formed, solution coating and 3-D printing.

TPU commercial products are available from a host of producers including: Estane®, Estaloc®, Isoplast®, Pearlthane®, Pearlbond®, Stat-Rite®, Pellethane™, Tecoflex™, Tecothane™, Carbothane™ TPUs from The Lubrizol Corporation; IROGRAN®, AVALON®, IROCOAT®, KRYSTALGRAN®, IROSTIC® TPUs from Huntsman Corporation; Desmopan®, Texin® TPUs from Covestro AG; Elastollan® TPUs from BASF SE; DIPRANE™, HYPERLAST™ TPUs from The Dow Chemical Company; EPACOL TK, EPALINE, EPAMOULD, Pakoflex from Epaflex Polyurethanes Spa; Edgetek™, Gravi-Tech™, NEUSoft™, OnFlex™ from PolyOne Corporation; Wanthan® TPUs from Wanhua Chemical Group Co. Ltd.; LARIPUR TPUs from Coim Group; Sheenthane® TPUs from Taiwan Sheen Soon; Miragnn® TPUs from Miragnn Chemical Company; Mirathane® TPUs from Miracil Chemicals; and Dryflex TPUs from Hexpol TPE. The various TPUs have broad use applications in a number of markets including: automotive, building & construction, film & sheet, engineering, footwear, synthetic leather & fibers, hose & tube, medical and wire & cable. It should be noted that most TPUs are processed without the need of solvents and thus the ability to form the TPU-I complex during the melting of the TPU is of high importance to obtain a commercial process which is simple to implement and is applicable to a large class of TPUs on the market. Though there can be interactions of iodine with the substructure chemical groups (ester groups, ether groups, carbonate groups, etc.), iodine strongly interacts and complexes with the urethane and urea groups in the TPU or PU matrixes.

Polyurethane dispersions (PUD) are a relatively new class of polyurethanes that were developed to reduce the environmental impact and healthcare risks of solventborne PU systems. PUD are finely dispersed PU in aqueous carrier that greatly reduces or eliminates the use of solvents when applying PU coatings.

The backbone of the PUD possesses the same basic composition as solvent-based PU polymers, but will contain hydrophilic groups that allows the polymer to be dispersed in water. The dispersed PU particles consist of a hydrophobic core surrounded by a hydrophilic shell composed of ionic groups and/or long-chain hydrophilic nonionic groups. These emulsifying groups are usually attached to the polymer backbone, but external emulsifiers and solvent can be used at times to facilitate the PUD. Ionic PUD incorporate ionic species into the PU backbone. For example, the use of chain extenders that contain sulfonate or carboxylate groups will result in an ionic PUD. Nonionic PUD often incorporate segments that contain longer segments of polyethylene oxide as the hydrophilic modifier. The aqueous PUD can be formulated into one-component and two-component formulations to generate coatings with high durability, good substrate adhesion, high water, stain and abrasion resistance, high toughness and corrosion protection. The aqueous coatings can be further formulated to undergo additional physical and chemical changes during drying of the coating to generate PU coatings that are less water sensitive and/or crosslinked to further enhance the resultant dry coating properties.
Thus, all PU, whether thermoplastics or thermosets or PUD, will form stable complexes with iodine as long as the iodine can be introduced in a homogeneous and controlled manner. Thus, any approach that can deliver and complex the iodine homogeneously and controlled within the PU matrix in a simple manufacturing process would be expected to have broad commercial appeal. A highly appropriate manner to form the TPU-I complex is when the TPU is in its “melt” physical form. A polymer melt occurs when a thermoplastic polymer is above its glass and/or crystallization temperatures and the polymer starts to behave as viscoelastic fluid and can be processed. It is during the polymer melt that the iodine complexation is initiated, formed and available for further downstream processing and product manufacture. There are two main approaches to obtain a TPU polymer melt: (1) by heating the TPU above its glass and/or crystallization temperatures or (2) by dissolving the TPU in a solvent.
Whereas a preferred approach to make the TPU-I complex involves heating and compounding above the TPU glass and/or crystallization temperatures without the use of solvents, there are specific applications in which TPUs can be processed and subsequently applied via solution coating. For such applications, the TPU-I can be made by adding the desired iodine source to the TPU during the dissolution step to make the TPU-I solution. The TPU-I solution is then processed as usual and the solvent removed to give the desired TPU-I complex. Whether the TPU-I complex is made via heating and compounding or via solution dissolution, the actual formation of the TPU-I complex is integrated into the normal and usual process steps necessary to handle the base TPU—there are no additional complex process steps needed to form the TPU-I complex.

Although TPUs elastomers have broad use application, crosslinked thermoset PU systems represents the largest group of PUs. The types of thermoset PU systems include: flexible PU foams, rigid PU foams, coatings, adhesives, sealants, elastomers, binders, reaction injection molding (RIM) and waterborne polyurethane dispersions. PU foams can be further classified as: open cell, reticulated or closed cell foams. For crosslinked thermoset PU systems, producing the PU-I complex cannot take place in the polymer melt, because PU thermosets do not melt at higher temperatures or dissolve in solvents. For this reason, the iodine source is suitably added and dissolved in one or more of the raw materials used to produce the PU thermoset, primarily the polyol, isocyanate and/or chain extender raw materials. Elemental iodine is very slightly soluble in water at 20° C. (1 g iodine in 3450 ml water), but shows much greater solubility in alcohols, amines and isocyanates. Higher concentrations of iodine in the PU raw materials can be prepared and subsequently reacted to give the final PU thermoset. The desired solubility in the PU raw materials preferably results in a minimum of 0.1 wt/wt % iodine loading in the final PU matrix.

It has also been discovered that the addition of an iodine source to the polyurethane reaction greatly inhibits the polyurethane reaction. However, this can be compensated by the addition of increased catalyst amounts to speed the reaction rate for commercial acceptability.

The preferred level of iodine loading in the PU-I complex is between 0.1-10 w % in the finished product, but higher levels of iodine are possible if subsequent dilution is conducted when using, for example, a master batch when processing TPU-I systems. The 0.1-10 w % iodine can be realized by using a combination of iodine sources, which includes elemental iodine, iodine salts and PVP-I. When using PVP-I, the preferable amount in the final (T)PU:PVP-I blend or finished product is in the range of 1-30% by wt., more preferably in the range of 1-20% by wt. Higher levels of incorporation of PVP-I are possible and not out of the scope of this invention. However, at high levels the mechanical properties of the (T)PU:PVP-I materials may become compromised and resultant complex materials behave more like PVP-I in their characteristics (e.g. slippery when wet, leaching of the PVP-I from the PU matrix).
There may be cases that this is desired in the production for example of slippery coatings, where the (T)PU:PVP:I is being “attached” to a substrate in which the bulk matrix is responsible for the mechanical integrity and the (T)PU:PVP-I coating is applied for both its lubricious properties and biocidal activity. High loading of PVP-I in a (T)PU:PVP-I thermoplastic or thermoset can also have the advantage of providing both a quick release of non-complexed PVP-I for quick and efficient biocidal/antiviral activity, followed by sustained biocidal/antiviral activity due to the complexed (T)PU:PVP-I. Thus, one can easily dial in varying biocidal/antiviral activity profiles based on the application by adjusting the ratio of PVP-I to (T)PU. This is of course also feasible to do with excess iodine loading to develop a non-complexed iodine phase, however, free iodine is corrosive, irritating to the skin and easily sublimes and thus the commercial usefulness of such systems are limited.
A further element of the invention described herein, is the discovery that the biocidal/antiviral activity of the PU-I complexes can be further tuned depending on the groups present in the PU matrix. Though iodine can complex with a host of different chemical groups, such as amide, urethane, urea groups, the actual complex energies of iodine with these various groups are different and in turn the resultant chemical group-iodine complexes show different activity profiles with respect to biocidal activity. Thus, due to the fact that the PU reaction can be tightly controlled, the iodine complexation with amide (via the addition of PVP-I or blending with nylons), urethane, urea, ester and ether groups can be tightly controlled by the raw materials used in the polymerization and/or processing to give resultant (T)PU-I systems with additional control over desired biocidal (e.g. bactericidal, fungicidal and viricidal) efficacy.
The PVP-I that may be used in the present invention to make PU—PVP-I blends can possess a broad range of available iodine content. Generally, the available iodine content is in the range of 1-25 w % and total iodine content is in the range of 2-35 w %. The production of such PVP-I systems is outlined in U.S. Pat. Nos. 2,706,701 and 2,900,305 and related patents. The use of PVP-I's with higher iodine content is not outside the scope of this invention and may be desired for certain applications. The PVP precursor used to make the PVP-I complex can be vinyl pyrrolidone homopolymers or copolymers having a K-value in the range of 10-60. The preferred PVP-I material to use for the production of PA-PVP-I blends is the PVP-I defined in the USP, EP and JP pharmacopeias having available iodine content of 9.0-12.0% and a nitrogen content of 9.5-11.5% and utilizes a precursor vinylpyrrolidone homopolymer of about K-value 30. This grade PVP-I is widely accepted, has a long use record, is readily available and is listed on the WHO's List of Essential Medicines.

It should be noted that preparation of blends, composites and/or interpenetrating networks (IPN) of the PU-I complexes with other natural and synthetic polymers, natural or synthetic fibers or fillers is not outside the scope of this invention. Such blends, composites and IPNs would be expected to also benefit from the biocidal activity of the PU-I complex. Also within the scope of this invention is the incorporation of one or more further biocidal agent(s) may contribute to enhancing the biocidal properties of the resulting biocidal material. For example, the addition of one or more further biocidal agent may further enhance the biocidal rate and/or activity against specific microorganism strains, while still possessing the broad-spectrum biocidal activity of the PU-I system. Suitable further biocidal agents include: silver, copper, gold, zinc metals and their salts; quaternary amino compounds (e.g. benzalkonium chloride and cetylpyridinium chloride); phenols and cresols; halophenols (e.g. p-chloro-m-xylenol); biguanides (e.g. chlorhexidine); anilides (e.g triclocarbon) and triclosan.

In addition, PU-I complexes with non-isocyanate polyurethane (NIPU) systems are within the scope of this invention. NIPUs are manufactured by one for the following four synthetic pathways: (1) the step-growth polymerization of bis-cyclic carbonates and amines, (2) the step-growth polymerization of linear activated dicarbonates and diamines, (3) the step-growth polymerization of linear activated dicarbomates and diols and (4) the ring-opening polymerization of cyclic carbomates. Due to the fact that NIPUs do not utilize isocyanates to form the PU, NIPUs are often considered more environmentally friendly and green than traditional polyol-isocyanates based PUs. Thus, there continues to be ongoing development to further advance NIPUs commercially. Though NIPUs do not utilizing isocyanates for their production, the urethane group is still generated in the NIPU polymer. This urethane group is able to complex with iodine in the same manner as traditional (T)PU systems and NIPU-I complexes can be formed by either dissolving the iodine in the NIPU thermoplastic melt via heating or solvent dissolution or can be dissolved in the starting raw materials used to form the NIPU thermoset.
Another advantage of the PU-I materials of this invention is that biocidal iodine is naturally occurring while the PU are readily hydrolysable. Thus, the PU-I systems are biodegradable by nature and/or can be recycled by reprocessing the PU-I scrap or chemical recycling of the PU-I by controlled chain cleavage via hydrolysis, glycolysis, alcoholysis or aminolysis to generate polyols, amines and iodine. Any iodine that does get into the environment can be readily metabolized by a host of living organisms (e.g. algae) or converted to natural iodine salts with little effect to the environment. Due to the readily available materials, low environmental “footprint”, good price structure and ease of manufacture, the PU-I materials of this invention have a broad product potential and customer acceptance. For this reason, the PU-I materials of this invention are expected to have broad use applications even for market segments that are highly cost sensitive. Such products include, but not limited to: water and air filters; foams for insulation, packaging and cushioning, building materials; carpet underlay; coatings; adhesives and sealants; binders; upholstered goods; footwear; automotive; elastomers.
The applications of the PU-I complex in finished products, blends, composites and IPNs outlined in this invention are vast. In theory, any potential use application that presently utilizes PU could be a potential use application for the PU-I materials. Applications that will especially benefit from this invention are applications that desire: reduced risk of microbial contamination, reduced risk of microbial growth, reduced risk of the spread of microbial contamination and the inactivation of viruses. For this reason, the PU-I systems should have broad use applications in various industrial, consumer, pharmaceutical, health, veterinarian and aquatic markets. Actual products include, but are not limited to: filters (both wet and dry; e.g. air filters for improved air quality and mouth caps for the protection of individuals from the spread of pathogenic microbes and viruses), medical device coatings, equipment or device housing, tapes, garments, foams and cushions (bed, couches), automotive, hard surface coatings (e.g. utensils, operating tables), dentistry articles (e.g. aspirators, tubes, chain elastics, restoration, cavity liner, etc.), building and construction materials (e.g. flooring, insulation, adhesives, coatings), carpet backing, medical devices, wound dressings, topical skin adhesive, operating and endo-scopes, catheters, tubes, breathing tubes, endotracheal tubes, intravascular catheters, deep intravenous lines, footwear, sponges, coatings, masks, synthetic sheets and fibers, textiles, food and device packaging, countertops, flexible surface coatings, key board covers, upholstery, artificial leather, tissue scaffolds, gloves, floor coatings, foams, adhesives, cardiac valves, pacemakers, breast implants, casting tapes, bone cement and adhesives, condoms, vaginal sponges, mats, cushioning, vibration and sound dampening applications and sealants.
All of the compositions, methods and experiments disclosed and claimed herein can be made and executed without undue experimentation in light of the present invention. While the compositions, methods and experiments of this invention have been described in terms of preferred embodiments, it will be apparent to those skilled in the art that variations may be applied to the compositions and methods and in the steps or in the sequence of steps of the method described herein without departing from the concept, spirit and scope of the invention. All modifications and applications apparent to those skilled in the art are deemed to be within the spirit, scope and concept of the invention as defined in the appended claims.

### Further Embodiments of the Invention

Further embodiment 1. A process to provide a biocidal polyurethane-iodine (PU-I) complex, comprising dissolving at least one iodine source into either (i) a thermoplastic polyurethane (TPU) or (ii) a polymerization mixture comprising raw materials for preparing a polyurethane thermoset; and allowing for formation of a biocidal PU-I complex.
Further embodiment 2. The process of further embodiment 1, comprising dissolving at least one the iodine source in a melt or solution of a thermoplastic polyurethane to provide a thermoplastic polyurethane-iodine (TPU-I) complex.
Further embodiment 3. The process of further embodiment 2, wherein said thermoplastic polyurethane comprises one or more of polyester-based, polyether-based, polycaprolactone-based, polyacrylate-based, aromatic and/or aliphatic thermoplastic polyurethanes.
Further embodiment 4. The process of further embodiment 1, comprising dissolving at least one iodine source in a polymerization mixture comprising (i) a polyol and (ii) isocyanate and/or small molecule chain extender commonly used in the fabrication of polyurethane to provide a thermoset polyurethane-iodine complex.
Further embodiment 5. The process of further embodiment 4, wherein the isocyanate comprises an aliphatic or an aromatic di- or multifunctional isocyanate.
Further embodiment 6. The process of further embodiment 4 or 5, wherein the polyol is selected from the group consisting of polyether polyol, polyester polyol, polycarbonate polyol, polycaprolactone polyol, polyacrylate polyol, and any combination thereof.
Further embodiment 7. The process of any one of the preceding further embodiments, wherein the at least one iodine source is selected from the group consisting of elemental iodine, polyvinylpyrrolidone-iodine (PVP-I), iodide salts, and combinations thereof.
Further embodiment 8. The process of further embodiment 6, wherein said PVP—I contains 1-25% available iodine and 2-35% total iodine.
Further embodiment 9. A biocidal polyurethane-iodine (PU-I) complex obtainable by a process according to any one of further embodiments 1-7.
Further embodiment 10. The PU-I complex of further embodiment 9, comprising 0.1-10 weight % elemental iodine.
Further embodiment 11. The polyurethane-iodine complex of further embodiment 9 or 10, comprising 1-30 weight % PVP-I.
Further embodiment 12. Use of a polyurethane-iodine complex according to any one of further embodiments 9-11 in the area of industrial, construction, consumer, pharmaceutical, health, veterinarian and/or aquatic markets.
Further embodiment 13. A biocidal product comprising a polyurethane-iodine complex according to any one of further embodiments 9-11.
Further embodiment 14. Biocidal product of further embodiment 13, selected from the group consisting of air filters, water and solution filters, mouth caps, gloves, equipment or device housing, adhesives, garments, curtains, fibers, hard surface coatings, dentistry articles, building materials, construction materials, carpets, medical devices, wound dressing, tissue scaffolds, operating and endo-scopes, catheters, tubes, breathing tubes, endotracheal tubes, intravascular catheters, deep intravenous lines, footwear, sponges, cutting planks, masks, hoses, food and device packaging, countertops, flexible surface coatings, key boards, upholstery, floor coatings, flooring, condoms, elastics, cardiac valves, pacemakers, mats, sealants, breast implants, implants, foams and gaskets.
Further embodiment 15. The use according to further embodiment 12, or a product according to further embodiment 13 or 14, comprising a blend, composite and/or interpenetrating network of the polyurethane-iodine complex with other natural or synthetic polymers, natural or synthetic fibers, biocidal agents and/or fillers.

### LEGEND TO THE FIGURES

FIG. 1: Schematic representation of exemplary processes for providing biocidal PU-I coatings by adding elemental iodine to an aqueous PU dispersion (PUD). (A) Iodine dissolved in a minimum amount of solvent is mixed with PUD. (B) Iodine added directly to aqueous PU dispersion. (C) Iodine dissolved in an excess amount of solvent is added to PUD. For details see Example 2.

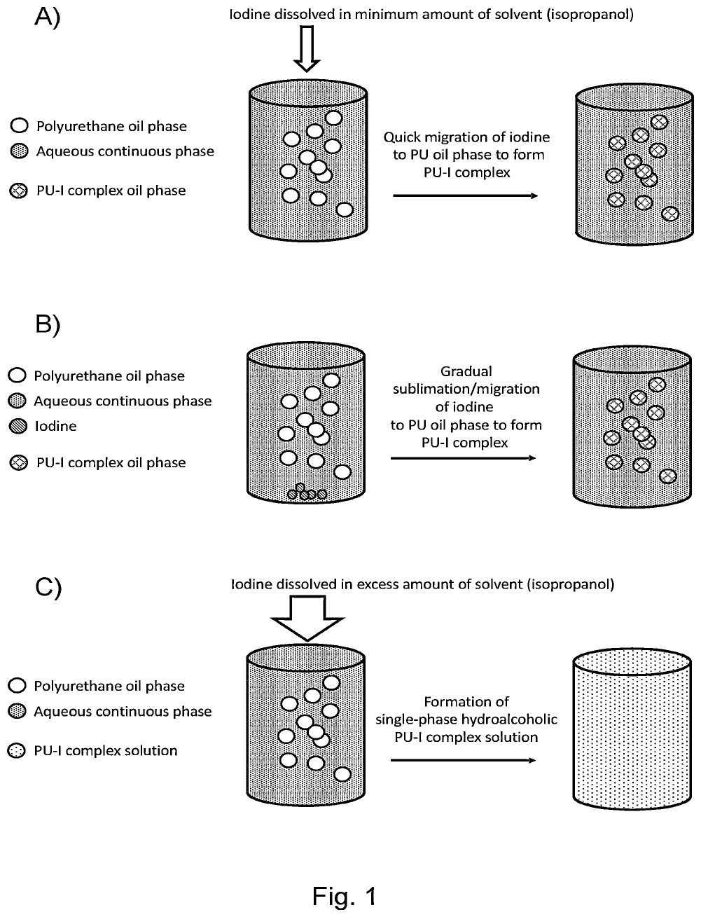

FIG. 2: Inhibitory effect of exemplary thermoplastic TPU-I filament samples on Staphylococcus aureus growth. For details see Example 4.

FIG. 3: Inhibitory effect of exemplary thermoset PU-I samples 6, 8 and 11 on (A) Candida albicans and (B) Streptococcus pyrogenes growth. For details see Example 4.

FIG. 4: Bactericidal effect of exemplary PU-I foams attached inside a human face mask. (A) Control PU Foam plated on growth media showing negligible biocidal activity. (B) PU-I Foam plated on growth media showing significant biocidal activity. For details see Example 5.

### EXPERIMENTAL SECTION

### EXAMPLE 1: Extrusion Studies with TPU

400 Gram extrusion studies were conducted in a Thermo Prism Eurolab 16 twin screw extruder having 16 mm screw diameter and 25 cm barrel length. The extruding barrel had five heating zones that were set at 200° C. The rotation speed of the screws was fixed at 400 rpm.

The raw materials used were an aliphatic polyether-based TPU (Lubrizol TecoFlex™ EG-93A-B30), EP pharmaceutical grade PVP-I (Boai NKY Pharmaceuticals Ltd. KoVidone®-I) and elemental iodine. The TPU was dried and grinded before use. Both PVP-I and elemental iodine were used as received. The powders were dry mixed in a mixer and then fed into the extruder. The extrudate in the form of a molten filament was cooled and pelletized. All extrusion experiments were conducted under a nitrogen atmosphere. The filaments and pellets were used “as is” or formed in sheets for subsequent bacterial growth studies.

TABLE 1
summary of the various extrusion
experiments conducted with TPU.

| | Wt. ratio of Ingredients | | | Biocidal | |
|---|---|---|---|---|---|
| Sample | TPU | PVP-I | Iodine | Activity | Observations |
| TPU1 | 100 | — | — | None | No biocidal activity |
| TPU2 | 95 | 5 | — | Yes | Polymer surface active |
| | | | | | against Staphylococcus |
| | | | | | aureus: minimal clearing |
| | | | | | zone |
| TPU3 | 92.5 | 5 | 2.5 | Yes | Polymer highly active |
| | | | | | against Staphylococcus |
| | | | | | aureus: surface active + |
| | | | | | large clearing zone |

### EXAMPLE 2: Producing PU-I Complex Coatings from Aqueous PUD

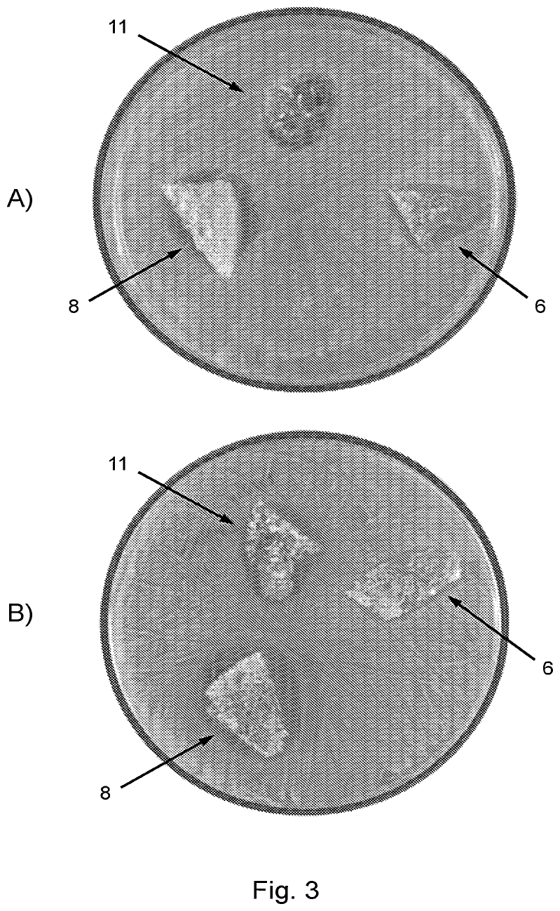

This example describes the manufacture of three different PU-I coating systems by adding elemental iodine as an iodine source into an aqueous PU dispersion (PUD). FIG. 3 provides a schematic drawing illustrating for each of the systems the migration of elemental iodine from the continuous phase to the dispersed PU phase to allow for PU-I complex formation.

### A) Iodine Dissolved in a Minimum Amount of Solvent.

A 5% (w/v) aqueous dispersion was prepared of RUCO-COAT EC 4811, which is a water based 32% w/v aliphatic nonionic PUD from Rudolf GmbH. 60 ml of the 5% aqueous solution containing 3 g polymer was stirred and to this dispersion was added 0.03 g elemental iodine dissolved in 2 ml isopropanol. The iodine quickly migrated to the polyurethane phase of the dispersion to generate a PU-I aqueous dispersion. The resultant opaque dispersion was milky brown in appearance and color. Because elemental iodine is not water-soluble, the dissolved iodine concentrates in the PU phase of the aqueous dispersion to generate the resultant PU-I dispersion. The resultant dispersion was stable and easily sprayable to coat desired objects. The final dried PU-I complex contained about 99% PU-1% iodine by weight.

### B) Iodine Added Directly to PU Dispersion

Solid elemental pearled iodine supplied by SQM Europe N. V. was added to the original (32 w/v %) RUCO-COAT EC 4811 aqueous dispersion. The actual amount of iodine added was 2% of the calculated polymer amount in the PUD. The iodine quickly sunk to the bottom of the dispersion to make a multi-phase system. The phases were mixed and the iodine was then allowed to sublime under controlled conditions at 50° C. for 12 hours which resulted in the migration of the elemental iodine to the PU phase of the dispersion to form the PU-I complex. The PU-I dispersion was further diluted with water to give a 17% solids PU-I aqueous dispersion. The resultant opaque dispersion was milky brown in appearance and color. This aqueous dispersion was stable and could be easily sprayed to coat desired objects. The final dried PU-I complex was about 98% PU-2% iodine by weight.

### C) Iodine Dissolved in an Excess Amount of Solvent.

The original 32% aqueous dispersion of RUCO-COAT EC4811 was diluted with an excess of isopropanol solution containing a small amount of dissolved iodine to give a soluble 5% PU-I hydro-alcoholic solution. The resultant light-brown solution was transparent and stable. The final dried complex was about 98.5% PU-1.5% iodine by weight.

### Biocidal Testing

Dispersions A and B and solution C comprising PU-I complex were applied via spray coating onto FFP2 medical face masks—both inside and outside surfaces. The coatings were allowed to dry at room temperature for 1 hr, followed by heated temperature drying at about 60° C. for an additional 30 minutes. The face masks were then worn for 3 hrs by volunteers, removed and the mask surfaces plated on nutrient agar for 24 hrs to observe the level of bacterial growth. All PU-I coatings resulted in a decrease in orally exhaled microbial growth when compared to control FFP2 face mask surfaces having no PU-I coating applied to the surface (data not shown).

### EXAMPLE 3: In-Situ PU-I Complex Formation (Polyurethane Thermoset Reactions)

The following raw materials were used to carry out the PU thermoset reactions:
Hexamethylene diisocyanate (HMDI)
Toluene diisocyanate (TDI)
Polyethylene glycol 400 (PEG)
Glycerol
Polyvinylpyrrolidone K17 (PVP)
1,4-diazabicyclo[2.2.2]octane (DABCO)
Povidone-iodine (PVP-I)
Elemental iodine (I₂)
   The thermoset PU polymerization reactions were conducted by the reaction of an isocyanate with polyethylene glycol/glycerol polyol blend with or without iodine source. If an iodine source was used in the reaction, the iodine was initially dissolved in the polyol blend prior to conducting the polyurethane reaction. Polyurethane reactions including an iodine source required the addition of a catalyst (DABCO) to accelerate the reaction. Without the addition of a catalyst, the polyurethane reactions containing an iodine source failed or required significantly higher curing temperatures and longer reaction times to form the thermoset.

The polyurethane reaction was conducted by reacting the isocyanate with polyol source at room temperature until homogeneous and the reaction mixture poured into molds and placed in a 60° C. oven for 2 hours. The resultant PU-I thermosets were then tested for biocidal activity. Table 2 presents a summary of the various compositions of polyurethane thermosets tested for biocidal activity.

TABLE 2

| | Isocyanate | | Polyol | | PVP-I | I₂ | Catalyst | Reaction | |
|---|---|---|---|---|---|---|---|---|---|
| Sample | HMDI | TDI | PEG/Glycerol | PVP | Wt % | Wt % | DABCO | Yes | No |
| PU2 | x | | x | | | | | x | |
| PU4 | x | | x | x | | | | x | |
| PU6 | | x | x | | | 2.0 | x | x | |
| PU7 | | x | x | | | 0.5 | x | x | |
| PU8 | | x | x | | 10.0 | 1.0 | x | x | |
| PU9 | | x | x | | | 1.0 | x | x | |
| PU10 | | x | x | x | | 1.0 | x | x | |
| PU11 | | x | x | | | 3.0 | x | x | |
| PU12 | | x | x | | 10.0 | | x | x | |
| PU16 | | x | x | | | | | x^{a} | |
| PU17 | x | | x | | 10.0 | | | | x |
| PU18 | x | | x | | | 0.5 | | | x^{b} |
| PU19 | | x | x | | | 0.5 | | | x^{b} |
| PU20 | x | | x | | 10.0 | | | | x |

^{a}Extremely fast reaction. No time to pour reaction mixture into mold.
^{b}Extremely slow reaction, after elevated temperatures and long reaction times, reaction did take place to some degree.

### EXAMPLE 4: Biocidal Activity of PU-I Complexes

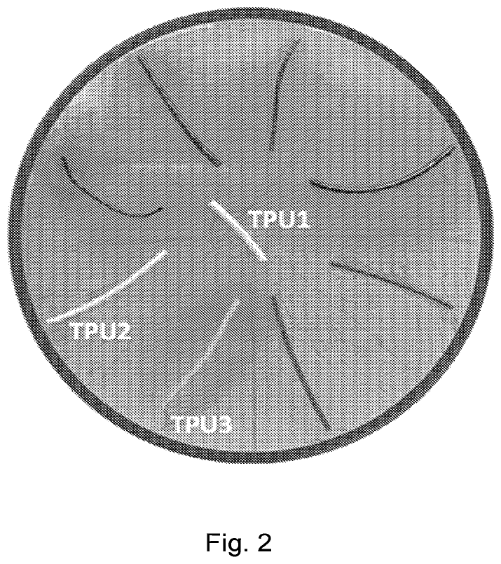

Bacterial testing on thermoplastic TPU-I samples from Example 1 and thermoset PU-I samples from Example 3 were conducted as follows: Staphylococcus aureus, Staphylococcus epidermis, Streptococcus pyrogenes and Candida albicans were grown in tryptic soy broth (TSB) overnight at 37° C. 100 μl of the overnight cultures were diluted to an optical density at 600 nm (OD₆₀₀) of 0.1 and subsequently plated on 100 mm Mueller-Hinton agar plates (MHA). TPU extruded filaments and PU thermosets were placed on top of the plates, and the plates were then incubated at 37° C. Growth inhibition was inspected at 24 and 48 h of incubation.
Exemplary TPU samples TPU2 and TPU3 showed biocidal activity versus S. aureus as shown in FIG. 2. Sample TPU3 containing 3 weight % iodine showed a significant clearing zone. Sample TPU2 containing 0.5 weight % iodine showed a significantly smaller clearing zone while the starting TPU1 sample showed no biocidal activity.
Exemplary PU samples PU6, PU8 and PU11 were highly active against S. aureus, S. epidermis, C. albicans and S. pyrogenes, showing defined clearing zones for all microorganisms tested. FIG. 3 shows the resultant clearing zones with respect to C. albicans and S. pyrogenes inhibition. Thermoset PU samples containing lower levels of iodine seem to be surface biocidal, but do not show the defined clearing zones as PU samples 6, 8 and 11. PU samples containing no iodine source were not biocidal.

### EXAMPLE 5: Manufacture and Biocidal Activity of PU-I Thermoset Foams

The following 3 PU-I foam systems were prepared and evaluated for biocidal (bactericidal and antiviral) activity.

| | | | Elemental | |
|---|---|---|---|---|
| | | Polyol/Chain | Iodine | Reaction |
| Sample | Isocyanate* | extender | (wt. %) | Details |
| PU21 | MDI | Polyester Polyol | 0.5 | Iodine |
| | | (CAS 28183-09-7) | | dissolved in |
| | | | | diol chain |
| | | | | extender, |
| | | | | surfactant and |
| | | | | tertiary amine |
| | | | | catalyst phase |
| PU22 | MDI | Polyether | 1.6 | Iodine |
| | | Polyol Mixture | | dissolved in |
| | | (CAS 9082-00-2 | | diol chain |
| | | and CAS | | extender, |
| | | 68650-94-2) | | surfactant and |
| | | | | tertiary amine |
| | | | | catalyst phase |
| PU23 | MDI | Polyether | 0.5 | Iodine |
| | | Polyol Mixture | | dissolved in |
| | | (CAS 9082-00-2 | | diol chain |
| | | and CAS | | extender, |
| | | 68650-94-2) | | surfactant and |
| | | | | tertiary amine |
| | | | | catalyst phase |

*MDI (Methylene diphenyl diisocyanate)

The PU21 sample foam was subsequently reticulated to form an open structure foam that could be used as a filter. Foams PU22 and PU23 were not reticulated.

### Bactericidal Testing:

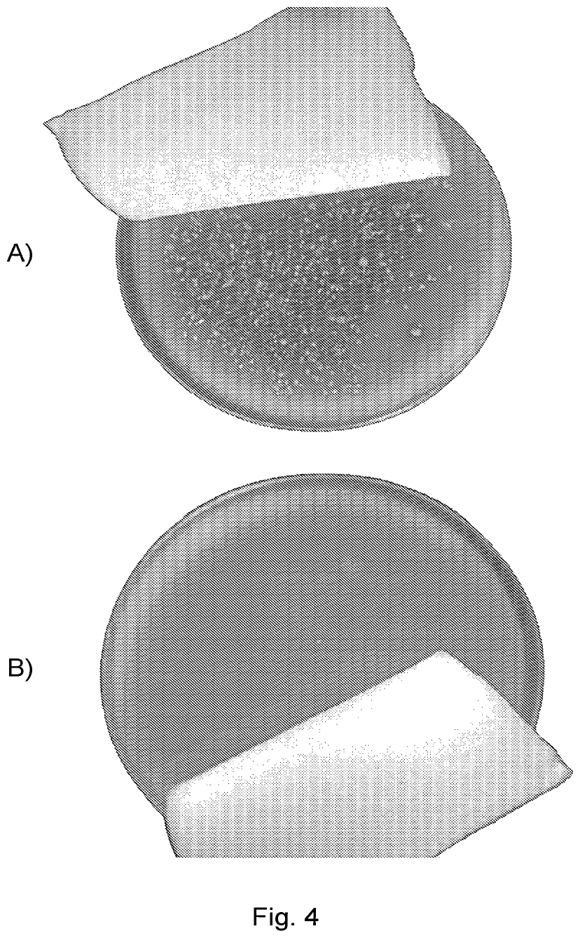

The reticulated PU21 foam sample was cut into 2 mm thick sheets that were further cut to shape to fit into a standard face mask. The PU21 foam was fastened to the inner side of the face mask and worn by a human volunteer for a period of 3 hours. The foam was then removed and plated on growth media overnight and the bacterial colonies observed. The same test was also conducted using a similar reticulated PU foam that contained no PU-I complex. FIG. 4 shows representative photos of the bacterial growth colonies for common PU reticulated foam and the PU21 PU-I complex foam. It demonstrates that the PU-I complex foam of the invention is highly biocidal against exhaled microorganisms.

### Antiviral Testing:

The antiviral activity was determined by adding a known amount of SARS-CoV-2 virus stock to the various foam samples (PU21-PU23). The virus stock was allowed to be in contact with the foam for the desired time period and then the viral supernatants were removed from the foam and titered onto 10,000 Vero E6 cells to quantitatively determine the viral titer reduction. PU foams with no iodine addition and no foam system were used as controls. Samples PU21, PU22 and PU23 showed significant viricidal activity against the SARS-CoV-2 virus. The three samples showed a minimum of 90% virus reduction at contact times of 10 minutes when compared against the control foam and no foam controls, and a minimum 99% virus reduction for 12 hours contact times.
A summary of the biocidal and antiviral properties is provided in the following table.

| | | | SARS-CoV-2 | SARS-CoV-2 |
|---|---|---|---|---|
| | | Exhaled | Viral Titer | Viral Titer |
| | | oral | Reduction vs | Reduction vs |
| Sample | Foam | bacteria | control foam** (%) | no foam** (%) |
| PU21 | Reticulated | Highly | 99.9/99.9 | 99.9/99.0 |
| | | biocidal | | |
| PU22 | Non- | Not tested | 99/99.9 | 99.0/99.0 |
| | reticulated | | | |
| PU23 | Non- | Not tested | 90.0/99.9 | 99/99.0 |
| | reticulated | | | |

**contact times 10 minutes/12 hours

### EXAMPLE 6: Difference Between Adding PVP-I as Dispersion or as Solution

This example provides comparative test results between PU foams prepared by adding PVP-I to the PU polymerization mixture either in the form of a dry powder (e.g. similar to U.S. Pat. No. 5,302,392) or as a solution in a raw material of the PU reaction (according to the in-situ process of the invention).

Two identical PU reactions were conducted in which the PVP-I was added as a powder or solution. The details are shown in the table below.

| | | | PVP-I | |
|---|---|---|---|---|
| Sample | Isocyanate* | Polyol System | (wt. %) | PVP-I |
| 1 | MDI | Polyester | 5 | Added as dry |
| | | Polyol/diol | | powder |
| 2 | MDI | Polyester | 5 | Added as solution |
| | | Polyol/diol | | |

*MDI (methylene diphenyl diisocyanate

Sample 1 was performed in accordance with U.S. Pat. No. 5,302,392 (see Example I). PVP-I powder was quickly dispersed in the polyester polyol to generate a uniform slurry of PVP-I powder in the polyol. Subsequently, the isocyanate MDI was added to initiate the polyurethane reaction. As expected, this reaction proceeded quickly to generate a PU foam in which the PVP-I complex powder was entrapped in the PU foam matrix. The PU reaction was not affected by the addition of the PVP-I powder because the PVP-I did not dissolve to any significant amount into the polyol and thus did not inhibit the polyurethane reaction. Consistent with the teaching of U.S. Pat. No. 5,302,392, the resultant product consisted primarily of PVP-I particles dispersed/entrapped in a PU foam matrix.
In the PVP-I solution reaction (sample 2), PVP-I was first dissolved in the chain extender diol to give a PVP-I solution. This single phase solution was mixed with the polyol to generate a polyol/diol PVP-I solution. The subsequent polyurethane reaction with MDI was conducted under the same reaction conditions as sample 1. However, this reaction was completely inhibited. Only after the addition of a significant amount of catalyst did the reaction proceed to give the homogeneous 1-phase PU-I complex foam.
As an additional control, a reaction was conducted in which PVP homopolymer (no iodine present) was dissolved in the chain extender diol to give a soluble PVP solution. The PVP solution was added to the polyol and the polyurethane reaction was conducted with MDI as in previous experiments. The polyurethane reaction proceeded as normal and was not inhibited, showing that the inhibition of the polyurethane reaction is caused by the iodine species.

1. A process to provide a biocidal polyurethane-iodine (PU-I) complex, comprising (i) dissolving at least one iodine source into one or more raw materials used for preparing a desired polyurethane (PU) to obtain a single phase iodine system, followed by (ii) conducting a PU polymerization reaction in the presence of the single phase iodine system to generate a biocidal PU-I complex in situ.

2. The process according to claim 1, wherein the at least one iodine source is selected from the group consisting of elemental iodine, polyvinylpyrrolidone-iodine (PVP-I), iodide salts, and any combination thereof.

3. The process according to claim 2, wherein the at least one iodine source is elemental iodine, optionally combined with PVP-I.

4. The process according to claim 2 or 3, wherein said PVP—I contains 1-25% available iodine and 2-35% total iodine.

5. The process according to any one of claims 1-4, comprising dissolving at least one iodine source in a polymerization mixture comprising (i) polyol; (ii) isocyanate; and (iii) a chain extender, crosslinker, catalyst, surfactant, solvent and/or additive used in the synthesis of the polyurethane to provide a thermoplastic or thermoset polyurethane-iodine complex.

6. The process according to any one of claims 1-4, comprising dissolving at least one iodine source in a polyol, a polyol blend, a low molecular weight alcohol with functionality 2, a low molecular weight amine with functionality ≥2 and/or solvent, followed by the addition of the desired isocyanate(s) to initiate the polyurethane reaction.

7. The process according to claim 5 or 6, wherein the isocyanate comprises an aliphatic di-, tri- or polyisocyanate, an aromatic di-, tri- or polyisocyanate, or any combination thereof.

8. The process according to any one of claims 5-7, wherein the polyol is selected from the group consisting of polyether polyol, polyester polyol, polycarbonate polyol, polycaprolactone polyol, polyacrylate polyol, and any combination thereof.

9. The process according to claim 5, wherein the chain extender is a low molecular weight diol or diamine, or any combination thereof.

10. The process according to claim 5, wherein the crosslinker is a low molecular weight alcohol or amine with a functionality of 2 or more.

11. The process according to any one of the preceding claims, comprising the use of a polyurethane catalyst, preferably a tertiary amine, metallic compound, or any combination thereof.

12. The process according to any one of the preceding claims, where the polyurethane polymerization reaction is conducted via multi-step, one-step bulk or solvent polymerization to form the final PU-I complex in pre-polymer formation stages or one process step.

13. A process to provide a biocidal polyurethane-iodine (PU-I) complex, comprising preparing a homogeneous mixture of (i) at least one iodine source and (ii) a thermoplastic polyurethane (TPU) or a polyurethane dispersion (PUD) to form a single-phase system that allows for formation of a biocidal PU-I complex.

14. The process according to claim 13, wherein the at least one iodine source is selected from the group consisting of elemental iodine, polyvinylpyrrolidone-iodine (PVP-I), iodide salts, and combinations thereof.

15. The process according to claim 14, wherein the at least one iodine source is or comprises elemental iodine.

16. The process of claim 13-15, comprising preparing a homogeneous single-phase system by blending at least one iodine source with TPU in a heated, molten or dissolved state.

17. The process of claim 16, comprising dissolving an iodine source in a TPU melt, followed by extrusion.

18. The process of claim 15, comprising adding elemental iodine to an aqueous PUD dispersion and allowing migration of the elemental iodine into the PU phase of the dispersion to obtain a homogeneous single-phase system wherein PU-I complex is formed as an aqueous PU-I dispersion.

19. Process according to claim 18, wherein elemental iodine is added to a PUD as a solution in a suitable solvent that dissolves elemental iodine and is compatible with the PU phase, preferably an alcohol, more preferably isopropanol.

20. Process according to claim 18, wherein elemental iodine is added as solid material, followed by iodine sublimation.

21. The process according to any one of claims 13-20, wherein said TPU or PUD comprises one or more of polyester-based, polyether-based, polycaprolactone-based, polyacrylate-based, aromatic and/or aliphatic thermoplastic polyurethanes.

22. The process according to any one of claims 13-21, wherein TPU or PUD makes up at least 85 weight %, preferably at least 90 weight %, more preferably at least 95 weight % of the total polymer content of the homogeneous mixture of PU and the at least one iodine source.

23. A biocidal polyurethane-iodine (PU-I) complex obtainable by a process according to any one of claims 1-22.

24. The PU-I complex of claim 23, comprising 0.1-10 weight % elemental iodine.

25. The PU-I complex according to claim 23 or 24, comprising 1-30 weight % PVP-I.

26. An aqueous dispersion or a solution comprising a biocidal PU-I complex according to any one of claims 23-24.

27. A biocidal coating comprising or consisting of a PU-I complex according to any one of claims 23-25.

28. Use of a polyurethane-iodine complex according to any one of claims 23-25 in the area of industrial, construction, consumer, pharmaceutical, health, veterinarian and/or aquatic markets.

29. A biocidal product comprising a polyurethane-iodine complex according to any one of claims 23-25.

30. A product provided with a biocidal coating according to claim 27.

31. Product according to claim 29 or 30, selected from the group consisting of air filters, water and solution filters, mouth caps, gloves, equipment or device housing, adhesives, garments, curtains, fibers, hard surface coatings, dentistry articles, building materials, construction materials, carpets, medical devices, wound dressing, tissue scaffolds, operating and endo-scopes, catheters, tubes, breathing tubes, endotracheal tubes, intravascular catheters, deep intravenous lines, footwear, sponges, cutting planks, masks, hoses, food and device packaging, countertops, flexible surface coatings, key boards, upholstery, floor coatings, flooring, condoms, elastics, cardiac valves, pacemakers, mats, mattresses, sealants, breast implants, implants, foams and gaskets.

32. The use according to claim 28, or a product according to claim 29-31, wherein the polyurethane-iodine complex forms a blend, composite and/or interpenetrating network with one or more other natural or synthetic polymers, natural or synthetic fibers, biocidal agents and/or fillers.
